(19) United States
(12) Patent Application Publication
(10) Pub. No.: US 2007/0003615 A1
(43) Pub. Date: Jan. 4, 2007

(54) Nanoparticulate clopidogrel and aspirin combination formulations

(75) Inventors: Scott Jenkins, Downingtown, PA (US); Gary G. Liversidge, West Chester, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/450,890

(22) Filed: Jun. 12, 2006

Related U.S. Application Data
(60) Provisional application No. 60/689,930, filed on Jun. 13, 2005.

(51) Int. Cl. A61K 9/20; A61K 31/60; A61K 31/4743

(52) U.S. Cl. 424/464; 514/165; 514/301; 977/906

(57) ABSTRACT

The present invention is directed to compositions comprising a nanoparticulate clopidogrel and aspirin combination, or salts or derivatives thereof, having improved clopidogrel bioavailability. The nanoparticulate clopidogrel particles, and optionally the nanoparticulate aspirin particles, of the composition have an effective average particle size of less than about 2000 nm and are useful in the prevention and treatment of pathologies induced by platelet aggregation. The clopidogrel and aspirin particles may also be formulated as a controlled release polymeric coating or matrix drug delivery system.

### CROSS-REFERENCE TO RELATED PATENT APPLICATIONS

This application claims the benefit under 35 U.S.C. § 119(e) of U.S. provisional application No. 60/689,930, filed on Jun. 12, 2005, which is incorporated by reference herein in its entirety.

### FIELD OF INVENTION

The present invention relates generally to compounds and compositions useful in the prevention and treatment of pathological states induced by platelet aggregation. More specifically, the invention relates to nanoparticulate clopidogrel combined with aspirin, optionally in a nanoparticulate form, or salts or derivatives thereof (referred to herein as “nanoparticulate clopidogrel and aspirin combination”), and compositions comprising the same. The nanoparticulate clopidogrel, and optionally the aspirin, within the combination compositions have an effective average particle size of less than about 2000 nm. The clopidogrel and/or aspirin particles may also be coated with any one of a number of polymeric materials for a controlled and/or delayed release formulation.

### BACKGROUND OF INVENTION

A. Background Regarding Clopidogrel

Clopidogrel is an inhibitor of platelet aggregation. Clopidogrel inhibits ADP-induced platelet aggregation by direct inhibition of adenosine diphosphate (ADP) binding to its receptor and of the subsequent ADP-mediated activation of the glycoprotein GPIIb/IIa complex. Clopidogrel also inhibits platelet aggregation induced by agonists other than ADP by blocking the amplification of platelet activation by released ADP.

The chemical name for clopidogrel bisulfate is methyl (+)-(S)-α-(2-chorophenyl)-6,7-dihydrothieno[3,2-c]pyridine-5(4H)-acetate sulfate (1:1). The empirical formula of clopidogrel bisulfate is C₁₆H₁₆ClNO₂S.H₂SO₄ and its molecular weight is 419.9. The structural formula is as follows:

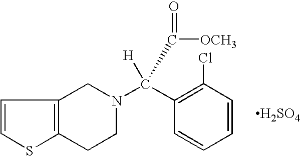

Clopidogrel bisulfate is a white to off-white powder. It is practically insoluble in water at neutral pH but is freely soluble at pH 1.0. It also dissolves freely in methanol, it dissolves sparingly in methylene chloride, and is practically insoluble in ethyl ether.

Clopidogrel bisulfate is commercially available under the trade name PLAVIX® by Bristol-Myers Squibb/Sanofi Pharmaceuticals Partnership (New York, N.Y.). PLAVIX® is administered as an oral tablet at a recommended dose of 75 mg once daily. PLAVIX® is provided as pink, round, biconvex, debossed film-coated tablets containing 97.875 mg of clopidogrel bisulfate which is the molar equivalent of 75 mg of clopidogrel base.

Clopidogrel bisulfate is indicated for the reduction of thrombotic events such as recent myocardial infarction (MI), recent stroke, or established arterial disease, and has been shown to reduce the rate of a combined end point of new ischemic stroke, new MI, and other vascular death. For patients with acute coronary syndrome, clopidogrel bisulfate has been shown to decrease the rate of a combined end point of cardiovascular death, MI, or stroke as well as the rate of a combined end point of cardiovascular death, MI, stroke, or refractory ischemia.

Clopidogrel has been described, for example, in U.S. Pat. Nos. 4,847,265 for “Dextro-Rotatory Enantiomer of Methyl Alpha-5 (4,5,6,7-Tetrahydro (3,2-c) Thieno Pyridyl) (2-Chlorophenyl)-Acetate and the Pharmaceutical Compositions Containing It”, 5,576,328 for “Method for the Secondary Prevention of Ischemic Events”, 5,989,578 for “Associations of Active Principles Containing Clopidogrel and an Anti-thrombotic Agent”, 6,429,210 and 6,504,030 both for “Polymorphic Clopidogrel Hydrogen Sulphate Form”, 6,635,763 for “Process to Prepare Clopidogrel”, 6,737,411 and 6,800,759 both for “Racemization and Enantiomer Separation of Clopidogrel”, and 6,858,734 for “Preparation of (S)-Clopidogrel and Related Compounds” These patents are hereby incorporated by reference.

Aspirin, also known as acetylsalicylic acid, is often used as an analgesic (against minor pains and aches), antipyretic (against fever), and anti-inflammatory. It has also an anticoagulant (blood thinning) effect and is used in long-term low-doses to prevent heart attacks.

Aspirin, CAS Number: 50-78-2, is chemically known as 2-acetoxybenzoic acid. Aspirin has a molecular formula of C₉H₈O₄ and a molecular weight of 180.16. The chemical structure of aspirin is shown below:

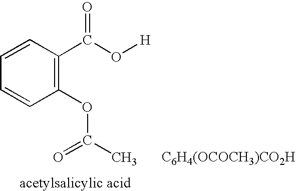

Aspirin is a colorless or white crystals or white crystalline powder or granule. It is odorless or almost odorless with a slight acid taste. Aspirin has a melting point of 136° C. (277° F.) and boiling point of 140° C. (284° F.). It is soluble 1 gm. in 300 of water, 1 in 5-7 gm./ml. in alcohol, 1 in 17 gm./ml. of chloroform and 1 in 20 gm./ml. of ether; soluble in solutions of acetates and citrates and, with decomposition, in solutions of alkali hydroxides and carbonates. It is incompatible with free acids, acetanilide, aminopyrine, phenazone, hexamine, iron salts, phenobarbitone sodium, quinine salts, potassium and sodium iodides, and alkali hydroxides, carbonates, and stearates. Acetylsalicylic acid is stable in dry air, but gradually hydrolyses in contact with moisture to acetic and salicylic acids. In solution with alkalis, the hydrolysis proceeds rapidly and the clear solutions formed may consist entirely of acetate and salicylate. Acetylsalicylic acid decomposes rapidly in solutions of ammonium acetate or of the acetates, carbonates, citrates or hydroxides of the alkali metals.

Aspirin is indicated as an analgesic for the treatment of mild to moderate pain, as an anti-inflammatory agent for the treatment of soft tissue and joint inflammation, and as an antipyretic drug. Aspirin is generally dosed in adults for pain and fever in amounts of 300-1000 mg every 4 hour for a maximum of 4 gram per day. For acute polyarthritis rheumatica, dosing is generally 1 gram given 6 times a day for a maximum of 8 grams a day. For rheumatoid arthritis, dosing is generally 0.5 grams to 1 gram given 6 times a day for a maximum of 8 grams a day. For prevention of transient ischaemic attacks and prevention of arterial thrombosis, dosing is generally 300 mg to 1200 mg a day in 2 or 3 doses.

Aspirin is used to lessen the chance of heart attack, stroke, or other problems that may occur when a blood vessel is blocked by blood clots. Aspirin helps prevent dangerous blood clots from forming. Low-dose long-term aspirin irreversibly blocks formation of thromboxane A2 in platelets, producing an inhibitory affect on platelet aggregation, and this blood thinning property makes it useful for reducing the incidence of heart attacks. Aspirin produced for this purpose often has strengths of 75 mg, 81 mg or 325 mg enteric coated tablets. High doses of aspirin are also given immediately after an acute heart attack.

Various brands of aspirin sold in the United States that include, for example, Acuprin 81, Amigesic, Anacin, Caplets, Anacin Maximum Strength, Anacin Tablets, Anaflex 750, Arthritis Pain Ascriptin, Arthritis Pain Formula, Arthritis Strength Bufferin, Arthropan, Aspergum, Aspirin Regimen Bayer Adult Low Dose, Aspirin Regimen Bayer Regular Strength Caplets, Aspir-Low, Aspirtab, Aspirtab-Max, Backache Caplets, Bayer Children's Aspirin, Bayer Select Maximum Strength Backache Pain Relief Formula, Bufferin Caplets, Bufferin Tablets, Buffex, Buffinol, Buffinol Extra, Cama Arthritis Pain Reliever, CMT, Cope, Disalcid, Doan's Regular Strength Tablets, Easprin, Ecotrin Caplets, Ecotrin Tablets, Empirin, Extended-release Bayer 8-Hour, Extra Strength Bayer Arthritis Pain Formula Caplets, Extra Strength Bayer Aspirin Caplets, Extra Strength Bayer Aspirin Tablets, Extra Strength Bayer Plus Caplets, Gensan, Genuine Bayer Aspirin Caplets, Genuine Bayer Aspirin Tablets, Halfprin, Healthprin Adult Low Strength, Healthprin Full Strength, Healthprin Half-Dose, Magan, Magnaprin, Marthritic, Maximum Strength Arthritis. Foundation Safety Coated Aspirin, Maximum Strength Ascriptin, Maximum Strength Doan's Analgesic Caplets, Mobidin. Mono-Gesic, Norwich Aspirin, P-A-C Revised Formula, Regular Strength Ascriptin, Salflex, Salsitab, Sloprin, St. Joseph Adult Chewable Aspirin, Tricosal, Trilisate, and ZORprin.

Aspirin has been described in numerous patents such as, for example, in U.S. Pat. No. 4,520,09 to Dunn for “Sustained Released Aspirin Formulation”; U.S. Pat. No. 4,716,042 to Blank et al. for “Stabilized Coated Aspirin Tablets”; U.S. Pat. No. 5,157,030 to Galat for “Rapidly Soluble Aspirin Compositions and Method”; U.S. Pat. No. 5,723,453 to Phykitt for “Stabilized, Water-Soluble Aspirin Composition”; and U.S. Reissued Pat. No. RE38,576 to Blahut for “Stabilized Aspirin Compositions and Method of Preparation for Oral and Topical Use”.

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of clopidogrel and aspirin combination.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using lodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate lododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” 6,432,381 for “Methods for Targeting Drug Delivery to the Upper and/or Lower Gastrointestinal Tract,” 6,592,903 for “Nanoparticulate Dispersions Comprising a Synergistic Combination of a Polymeric Surface Stabilizer and Dioctyl Sodium Sulfosuccinate,” 6,582,285, for “Apparatus for sanitary wet milling;” 6,656,504 for “Nanoparticulate Compositions Comprising Amorphous Cyclosporine;” 6,742,734 for “System and Method for Milling Materials;” 6,745,962 for “Small Scale Mill and Method Thereof;” 6,811,767 for “Liquid droplet aerosols of nanoparticulate drugs;” and 6,908,626 for “Compositions having a combination of immediate release and controlled release characteristics;” 6,969,529 for “Nanoparticulate compositions comprising copolymers of vinyl pyrrolidone and vinyl acetate as surface stabilizers;” 6,976,647 for “System and Method for Milling Materials,” all of which are specifically incorporated by reference.

In addition, U.S. Patent Application No. 20020012675 A1, published on Jan. 31, 2002, for “Controlled Release Nanoparticulate Compositions,” U.S. Patent Publication No. 20050276974 for “Nanoparticulate Fibrate Formulations;” U.S. Patent Publication No. 20050238725 for “Nanoparticulate compositions having a peptide as a surface stabilizer;” U.S. Patent Publication No. 20050233001 for “Nanoparticulate megestrol formulations;” U.S. Patent Publication No. 20050147664 for “Compositions comprising antibodies and methods of using the same for targeting nanoparticulate active agent delivery;” U.S. Patent Publication No. 20050063913 for “Novel metaxalone compositions;” U.S. Patent Publication No. 20050042177 for “Novel compositions of sildenafil free base;” U.S. Patent Publication No. 20050031691 for “Gel stabilized nanoparticulate active agent compositions;” U.S. Patent Publication No. 20050019412 for “Novel glipizide compositions;” U.S. Patent Publication No. 20050004049 for “Novel griseofulvin compositions;” U.S. Patent Publication No. 20040258758 for “Nanoparticulate topiramate formulations;” U.S. Patent Publication No. 20040258757 for “Liquid dosage compositions of stable nanoparticulate active agents;” U.S. Patent Publication No. 20040229038 for “Nanoparticulate meloxicam formulations;” U.S. Patent Publication No. 20040208833 for “Novel fluticasone formulations;” U.S. Patent Publication No. 20040195413 for “Compositions and method for milling materials;” U.S. Patent Publication No. 20040156895 for “Solid dosage forms comprising pullulan;” U.S. Patent Publication No. U.S. Patent Publication No. U.S. Patent Publication No. 20040156872 for “Novel nimesulide compositions;” U.S. Patent Publication No. 20040141925 for “Novel triamcinolone compositions;” U.S. Patent Publication No. 20040115134 for “Novel nifedipine compositions;” U.S. Patent Publication No. 20040105889 for “Low viscosity liquid dosage forms;” U.S. Patent Publication No. 20040105778 for “Gamma irradiation of solid nanoparticulate active agents;” U.S. Patent Publication No. 20040101566 for “Novel benzoyl peroxide compositions;” U.S. Patent Publication No. 20040057905 for “Nanoparticulate beclomethasone dipropionate compositions;” U.S. Patent Publication No. 20040033267 for “Nanoparticulate compositions of angiogenesis inhibitors;” U.S. Patent Publication No. 20040033202 for “Nanoparticulate sterol formulations and novel sterol combinations;” U.S. Patent Publication No. 20040018242 for “Nanoparticulate nystatin formulations;” U.S. Patent Publication No. 20040015134 for “Drug delivery systems and methods;” U.S. Patent Publication No. 20030232796 for “Nanoparticulate polycosanol formulations & novel polycosanol combinations;” U.S. Patent Publication No. 20030215502 for “Fast dissolving dosage forms having reduced friability;” U.S. Patent Publication No. 20030185869 for “Nanoparticulate compositions having lysozyme as a surface stabilizer;” U.S. Patent Publication No. 20030181411 for “Nanoparticulate compositions of mitogen-activated protein (MAP) kinase inhibitors;” U.S. Patent Publication No. 20030137067 for “Compositions having a combination of immediate release and controlled release characteristics;” U.S. Patent Publication No. 20030108616 for “Nanoparticulate compositions comprising copolymers of vinyl pyrrolidone and vinyl acetate as surface stabilizers;” U.S. Patent Publication No. 20030095928 for “Nanoparticulate insulin;” U.S. Patent Publication No. 20030087308 for “Method for high through put screening using a small scale mill or microfluidics;” U.S. Patent Publication No. 20030023203 for “Drug delivery systems & methods;” U.S. Patent Publication No. 20020179758 for “System and method for milling materials; and U.S. Patent Publication No. 20010053664 for “Apparatus for sanitary wet milling,” describe nanoparticulate active agent compositions and are specifically incorporated by reference.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

Clopidogrel and aspirin combination has high therapeutic value in the prevention and treatment of pathologies induced by platelet aggregation. However, because clopidogrel is practically insoluble in water, significant bioavailability can be problematic. There is a need in the art for nanoparticulate clopidogrel and aspirin combination formulations which overcome this and other problems associated with the use of clopidogrel and aspirin combination in the prevention and treatment of pathologies induced by platelet aggregation. The present invention satisfies this need.

### SUMMARY OF THE INVENTION

The present invention relates to compositions comprising clopidogrel, or salts or derivatives thereof. The invention further relates to nanoparticulate compositions comprising a clopidogrel or salts or derivatives thereof, and compositions comprising a clopidogrel and aspirin combination, or salts or derivatives thereof. The compositions comprise nanoparticulate clopidogrel and, optionally nanoparticulate aspirin particles, and at least one surface stabilizer adsorbed or associated with the surface of the clopidogrel and aspirin combination particles. The nanoparticulate clopidogrel particles have an effective average particle size of less than about 2,000 nm. Optionally, nanoparticulate aspirin particles have an effective average particle size of less than about 2,000 nm.

Conventional clopidogrel bisulfate tablets have limited bioavailability because the drug is practically insoluble in water. The present invention provides improved dissolution rate of clopidogrel bisulfate that would result in enhanced bioavailability allowing a smaller dose to give the same in vivo blood levels. Additionally, clopidogrel bisulfate becomes soluble when exposed to the low pH environment of the stomach and then precipitates from solution when the drug enters the higher pH region of the proximal small intestine. This mechanism limits the bioavailability of clopidogrel bisulfate. Applying an enteric coating to the clopidogrel bisulfate formulation would stop the solubilization followed by precipitation from occurring, which would increase the bioavailability. As clopidogrel bisulfate can cause significant gastric irritation (e.g., to the esophagus and stomach) it is expected that an enteric coated formulation would have decreased gastric irritancy by not having the drug dissolved in the stomach. Accordingly, the present invention includes an enteric coated clopidogrel composition, such as for example, clopidogrel bisulfate, an enteric coated nanoparticulate clopidogrel composition, and an enteric coated combination of nanoparticulate clopidogrel and aspirin particles.

The present invention then, relates to compositions comprising clopidogrel, nanoparticulate clopidogrel, and nanoparticulate clopidogrel and aspirin combination, or salts or derivatives thereof, for the treatment of cardiovascular disease. Moreover, the present invention further comprises a nanoparticulate clopidogrel and aspirin combination particles having one or both actives, clopidogrel and aspirin, coated with one or more polymeric coatings for a sustained and/or delayed controlled drug release.

The present invention includes the administration of clopidogrel bisulfate as a multiparticulate formulation that minimizes high local concentrations of dissolved drug in the gastro-intestinal tract which would be expected to minimize gastro-intestinal irritancy. Therefore, the invention also encompasses a multiparticulate formulation of clopidogrel bisulfate.

The present invention further includes coadministration clopidogrel with aspirin to enhance the therapeutic outcome of clopidogrel bisulfate. The aspirin component can also be, but it not necessarily, a nanoparticulate formulation to enhance dissolution. The aspirin component is preferably enteric coated and in a multiparticulate form to decrease aspirin's gastrointestinal irritancy.

The invention is useful in improving bioavailability and therefore therapeutic outcome for all treatments requiring clopidogrel bisulfate and aspirin, including but not limited to, reduction of thrombotic events.

The present invention also relates to a controlled release formulation in which the nanoparticulate clopidogrel and aspirin combination particles are coated with one or more polymeric coatings or incorporated in a polymeric material matrix so that the active is released at a sustained and/or delayed rate of release for an improved, more consistent dissolution rate within the stomach and small intestines thereby avoiding the occurrence of localized “hot spots” of high drug concentrations.

Enteric-coated pharmaceutical tablet compositions are known. Enteric coated tablets provide resistance to disintegration at low pH levels while releasing drugs at higher pHs. The nanoparticulate clopidogrel or clopidogrel and aspirin combination particles of the present invention are preferably enterically coated to delay the release of the clopidogrel and/or aspirin from orally ingestible dosage forms. In particular, by using an enteric coating, solubilization and precipitation of the clopidogrel active agent of the present invention is prevented. Stomach irritancy is also decreased, particularly with aspirin also enterically. Representatively, most enteric coating polymers become soluble at pH 5.5 and above, with maximum solubility rates at pHs greater than 6.5. Numerous enteric coated and/or extended release pharmaceutical compositions and the methods of making these compositions have been disclosed in the art. They may include extra ingredients in addition to the active pharmaceutical ingredient, such as fillers, buffering agents, binders and wetting agents, as desired for a certain composition. Enteric coatings allow delivery of the active agent(s) to a specific location within the body, e.g., delivery in the lower GI tract, i.e., in the colon or the upper intestines, i.e., the duodenum of the small intestine. For example, in some embodiments, no more than about 0.05%, no more than about 0.5%, no more than about 1%, no more than about 5% no more than about no more than about 10%, no more than about 20%, or no more than about 30% of the active agent (e.g., clopidogrel and/or aspirin) of the enteric coated compositions of the invention dissolves in the stomach of a subject, relative to the total dose administered to the subject. In other embodiments, at least about 50%, at least about 60%, at least about 70%, at least about 80%, at least about 90%, at least about 95%, at least about 97%, or at least about 100% of the active agent (e.g., clopidogrel and/or aspirin) is released in the intestine of a subject, relative to the total dose administered to the subject. The enteric coat may include one or more materials that remain intact during the period of time that the tablet resides in the stomach and do not dissolve, disintegrate, or change structural integrity in the stomach. Preferably, the clopidogrel compound of the present invention includes a delayed-release methodology such as that described in Pharmaceutical Dosage Forms and Drug Delivery Systems, “Modified-Release Dosage Forms and Drug Delivery Systems”, Lippincott Williams & Wilkins, 1999, Chapter 8, pp. 229-244, the disclosure of which is herein incorporated by reference in its entirety. As described therein, a delayed-release form provided is designed to release the drug from the dosage from at a time other than promptly after administration. The coating is non-toxic and preferably includes any pharmaceutically acceptable enteric polymer that is predominantly soluble in the intestinal fluid, but substantially insoluble in the gastric juices. A wide variety of other polymeric materials are known to possess such solubility properties.

The nanoparticulate clopidogrel and aspirin combination particles can also be formulated as an intravenous solution for administration immediately prior to or during a cardiac event for the immediate onset of drug therapeutic action as well as improved ease of administration.

A preferred dosage form of the invention is a solid dosage form, although any pharmaceutically acceptable dosage form can be utilized.

Another aspect of the invention is directed to pharmaceutical compositions comprising a nanoparticulate clopidogrel and aspirin combination, or salts or derivatives thereof, and at least one surface stabilizer, a pharmaceutically acceptable carrier, as well as any desired excipients.

Another embodiment of the invention is directed to nanoparticulate clopidogrel and aspirin combination compositions comprising one or more additional compounds useful in the prevention and treatment of a pathological state induced by platelet aggregation, preferably cardiovascular disease.

This invention further discloses a method of making the inventive nanoparticulate clopidogrel and aspirin combination composition. Such a method comprises contacting the nanoparticulate clopidogrel and aspirin combination, or salts or derivatives thereof, with at least one surface stabilizer for a time and under conditions sufficient to provide a stabilized nanoparticulate clopidogrel and aspirin combination composition.

The present invention is also directed to methods of treatment including but not limited to, the prevention and treatment of pathological states induced by platelet aggregation, preferably cardiovascular disease, using the novel nanoparticulate clopidogrel and aspirin combination compositions disclosed herein. Such methods comprise administering to a subject a therapeutically effective amount of a nanoparticulate clopidogrel and aspirin combination, or salts or derivatives thereof. Other methods of treatment using the nanoparticulate compositions of the invention are known to those of skill in the art.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

I. Nanoparticulate Clopidogrel and Aspirin Combination Compositions

The present invention is directed to nanoparticulate compositions comprising a clopidogrel and aspirin combination, or salts or derivatives thereof. The compositions comprise a clopidogrel and aspirin combination, or salts or derivatives thereof, and preferably at least one surface stabilizer adsorbed on the surface of the drug. The clopidogrel and aspirin combination, or salts or derivatives thereof, particles have an effective average clopidogrel particle size of less than about 2000 nm.

Advantages of the nanoparticulate clopidogrel and aspirin combination formulations of the invention include, but are not limited to: (1) smaller tablet or other solid dosage form size; (2) smaller doses of drug required to obtain the same pharmacological effect as compared to conventional microcrystalline forms of clopidogrel and aspirin; (3) increased bioavailability as compared to conventional microcrystalline forms of clopidogrel; (4) improved pharmacokinetic profiles; (5) an increased rate of dissolution for the clopidogrel as compared to conventional microcrystalline forms of the same clopidogrel; (6) the clopidogrel and aspirin combination compositions can be used in conjunction with other active agents useful in the prevention and treatment of pathologies induced by platelet aggregation; and (7) decreased gastrointestinal irritancy resulting from enterically coated clopidogrel active agent and/or aspirin.

The present invention also includes nanoparticulate clopidogrel and aspirin combinations, or salts or derivatives thereof, compositions together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, ocular, local (powders, ointments, or drops), buccal, intracisternal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

The present invention is described herein using several definitions, as set forth below and throughout the application.

The term “effective average particle size of less than about 2000 nm,” as used herein, means that at least about 50% of the nanoparticulate clopidrogrel particles (or aspirin particles) have a size of less than about 2000 nm, by weight (or by other suitable measurement technique, such as by number, volume, etc.) when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable clopidrogrel nanoparticulate particles, and stable aspirin nanoparticulate particles, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the clopidrogrel or aspirin has not been subject to a heating step at or above the melting point of the clopidrogrel or aspirin in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

II. Preferred Characteristics of the Nanoparticulate Clopidogrel and Aspirin Combinations of the Invention

A. Increased Bioavailability

The compositions of the invention comprising a nanoparticulate clopidogrel and aspirin combination, or salts or derivatives thereof, are proposed to exhibit increased bioavailability of the clopidogrel, and require smaller doses as compared to prior conventional clopidogrel formulations. In one embodiment of the invention, the nanoparticulate clopidogrel composition, in accordance with standard pharmacokinetic practice, has a bioavailability that is about 50% greater than a conventional dosage form, about 40% greater, about 30% greater, about 20% greater, or about 10% greater.

B. Improved Pharmacokinetic Profiles

The nanoparticulate clopidogrel and aspirin combination, or salts or derivatives thereof, formulations of the invention are proposed to exhibit improved pharmacokinetic profiles in which the maximum plasma concentration of clopidogrel are higher for a given dose than those occurring following administration of a conventional dosage form. In addition, the time to reach maximum plasma concentration will be shorter with nanoparticulate clopidogrel. These changes will improve the therapeutic efficacy of clopidogrel.

The invention preferably provides compositions comprising at least one nanoparticulate clopidogrel or derivative or a salt thereof, and optionally either conventional microcrystalline or nanoparticulate aspirin, having a desirable pharmacokinetic profile when administered to mammalian subjects. The desirable pharmacokinetic profile of the compositions of the invention preferably includes, but is not limited to: (1) a Cₘₐₓ for the clopidogrel or derivative or a salt thereof, when assayed in the plasma of a mammalian subject following administration, that is preferably greater than the Cₘₐₓ for a non-nanoparticulate formulation of the same clopidogrel administered at the same dosage; and/or (2) an AUC for the clopidogrel or derivative or a salt thereof, when assayed in the plasma of a mammalian subject following administration, that is preferably greater than the AUC for a non-nanoparticulate formulation of the same clopidogrel administered at the same dosage; and/or (3) a Tₘₐₓ for the clopidogrel or derivative or a salt thereof, when assayed in the plasma of a mammalian subject following administration, that is preferably less than the Tₘₐₓ for a non-nanoparticulate formulation of the same clopidogrel administered at the same dosage.

The invention also encompasses compositions comprising nanoparticulate aspirin and providing: (1) a Cₘₐₓ for aspirin or a salt or derivative thereof, when assayed in the plasma of a mammalian subject following administration, that is preferably greater than the Cₘₐₓ for a non-nanoparticulate formulation of the aspirin, administered at the same dosage; and/or (2) an AUC for aspirin or a salt or derivative thereof, when assayed in the plasma of a mammalian subject following administration, that is preferably greater than the AUC for a non-nanoparticulate formulation of the aspirin, administered at the same dosage; and/or (3) a Tₘₐₓ for aspirin or a salt or derivative thereof, when assayed in the plasma of a mammalian subject following administration, that is preferably less than the Tₘₐₓ for a non-nanoparticulate formulation of the same aspirin administered at the same dosage.

For example, in one embodiment, a composition comprising a nanoparticulate clopidogrel or a derivative or salt thereof, and at least one surface stabilizer exhibits in comparative pharmacokinetic testing with a non-nanoparticulate formulation of the same clopidogrel, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate clopidogrel formulation.

In another embodiment, a composition comprising a nanoparticulate clopidogrel or a derivative or salt thereof, and at least one surface stabilizer exhibits in comparative pharmacokinetic testing with a non-nanoparticulate formulation of the same clopidogrel, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate clopidogrel formulation.

In another embodiment, a composition comprising a nanoparticulate clopidogrel or a derivative or salt thereof, and at least one surface stabilizer exhibits in comparative pharmacokinetic testing with a non-nanoparticulate formulation of the same clopidogrel administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate clopidogrel formulation.

The desirable pharmacokinetic profile, as used herein, is the pharmacokinetic profile measured after the initial dose of the clopidogrel or derivative or a salt thereof.

C. The Pharmacokinetic Profiles of the Clopidogrel/Aspirin Compositions of the Invention are not Affected by the Fed or Fasted State of the Subject Ingesting the Compositions

The invention encompasses compositions comprising a nanoparticulate clopidogrel and aspirin, or a derivative or a salt thereof, wherein the pharmacokinetic profile of clopidogrel, and optionally aspirin, is not substantially affected by the fed or fasted state of a subject ingesting the composition. This means that there is no substantial difference in the quantity of drug absorbed or the rate of drug absorption when the nanoparticulate clopidogre1/aspirin compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed.

D. Bioequivalency of Clopidogrel/Aspirin Compositions of the Invention When Administered in the Fed Versus the Fasted State

The invention also provides compositions comprising a nanoparticulate clopidogrel and aspirin, or a derivative or a salt thereof, in which administration of the composition to a subject in a fasted state is bioequivalent to administration of the composition to a subject in a fed state.

The difference in absorption of the clopidogrel/aspirin compositions of the invention, when administered in the fed versus the fasted state (absorption of clopidogrel, aspirin, or a combination thereof), preferably is less than about 100%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In one embodiment of the invention, the invention encompasses compositions comprising at least one nanoparticulate clopidogrel and aspirin, which can also be in a nanoparticulate size, wherein administration of the composition a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA) (Cₘₐₓ and AUC for clopidogrel, aspirin, or a combination thereof). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cmax must between 0.70 to 1.43.

E. Dissolution Profiles of the Clopidogrel and Aspirin Combinations of the Invention

The compositions of the invention comprising nanoparticulate clopidogrel and aspirin combination, or salts or derivatives thereof, are proposed to have unexpectedly dramatic dissolution profiles. Rapid dissolution of an administered active agent is preferable, as faster dissolution generally leads to faster onset of action and greater bioavailability. To improve the dissolution profile and bioavailability of the clopidogrel and aspirin combination it would be useful to increase the drug's dissolution so that it could attain a level close to 100%.

The clopidogrel component of the invention preferably has a dissolution profile in which within about 5 minutes at least about 20% of the composition is dissolved. In other embodiments of the invention, at least about 30% or at least about 40% of the clopidogrel composition is dissolved within about 5 minutes. In yet other embodiments of the invention, preferably at least about 40%, at least about 50%, at least about 60%, at least about 70%, or at least about 80% of the clopidogrel composition is dissolved within about 10 minutes. Finally, in another embodiment of the invention, preferably at least about 70%, at least about 80%, at least about 90%, or at least about 100% of the clopidogrel composition is dissolved within 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

F. Redispersibility of the Clopidogrel and Aspirin Combination Compositions of the Invention

An additional feature of the compositions comprising a clopidogrel and aspirin combination, or salts or derivatives thereof, is that the compositions redisperse such that the effective average particle size of the redispersed clopidogrel particles, aspirin particles, or a combination thereof is less than about 2 microns. This is significant, as if upon administration the clopidogrel and aspirin combination compositions of the invention did not redisperse to a substantially nanoparticulate size, then the dosage form may lose the benefits afforded by formulating the clopidogrel and aspirin combination into a nanoparticulate size.

This is because nanoparticulate active agent compositions benefit from the small particle size of the active agent; if the active agent does not disperse into the small particle sizes upon administration, them “clumps” or agglomerated active agent particles are formed, owing to the extremely high surface free energy of the nanoparticulate system and the thermodynamic driving force to achieve an overall reduction in free energy. With the formulation of such agglomerated particles, the bioavailability of the dosage form my fall well below that observed with the liquid dispersion form of the nanoparticulate active agent.

Moreover, the nanoparticulate clopidogrel/aspirin compositions exhibit dramatic redispersion of the nanoparticulate clopidogrel particles, aspirin particles, or a combination thereof upon administration to a mammal, such as a human or animal, as demonstrated by reconstitution/redispersion in a biorelevant aqueous media such that the effective average particle size of the redispersed clopidogrel particles, aspirin particles, or a combination thereof is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed clopidogrel particles, aspirin particles, or a combination thereof (redispersed in water, a biorelevant media, or any other suitable liquid media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

G. Clopidogrel and Aspirin Combination Compositions Used in Conjunction with Other Active Agents

The compositions comprising a clopidogrel and aspirin combination, or salts or derivatives thereof, can additionally comprise one or more compounds useful in the prevention and treatment of pathologies induced by platelet aggregation, or the clopidogrel and aspirin combination compositions can be administered in conjunction with such a compound. Examples of such compounds include, but are not limited to calcium-entry blocking agents, antianginal agents, cardiac glycosides, vasodilators, antihypertensive agents, blood lipid-lowering agents, antidysrhythmic agents, and antithrombotic agents.

H. Reduced Gastrointestinal Irritancy with Enterically Coated Clopidogrel and/or Aspirin Combination Compositions of the Invention

An additional feature of the compositions of the invention is that the compositions may advantageously be enterically or film coated to reduce gastrointestinal irritancy of the patient (e.g., irritation of the stomach and/or esophagus). For example, in some embodiments, a solid dose form comprising a clopidogrel, or salts or derivatives thereof, may be enterically or film coated. In other embodiments, a solid dose form comprising a clopidogrel and aspirin combination, or salts or derivatives thereof, may be enterically or film coated.

Enteric coatings allow delivery of the active agent(s) to a specific location within the body, e.g., delivery in the lower GI tract, i.e., in the colon, or the upper intestines, i.e., the duodenum of the small intestine, and may act to prevent or inhibit delivery of active agent(s) to the stomach. For example, in some embodiments, no more than about 0.05%, no more than about 0.5%, no more than about 1% no more than about 5%, no more than about 10%, no more than about 20%, no more than about 30%, or no more than about 40% of the active agent (e.g., clopidogrel and/or aspirin) of the enteric coated compositions of the invention dissolves in the stomach of a subject, relative to the total dose administered to the subject. In other embodiments, at least about 50%, at least about 60%, at least about 70%, at least about 80%, at least about 90%, at least about 95%, at least about 97% or at least about 100% of the active agent (e.g., clopidogrel and/or aspirin) is released in the intestine of a subject, relative to the total dose administered to the subject.

Examples of suitable film-coating polymers include enteric polymer coating materials, such as, for example, cellulose acetate phthalate, cellulose acetate trimaletate, hydroxypropyl methylcellulose phthalate, polyvinyl acetate phthalate, Eudragit® poly acrylic acid and poly acrylate and methacrylate coatings, polyvinyl acetaldiethylamino acetate, hydroxypropyl methylcellulose acetate succinate, cellulose acetate trimellitate, shellac; hydrogels and gel-forming materials, such as, for example, carboxyvinyl polymers, sodium alginate, sodium carmellose, calcium carmellose, sodium carboxymethyl starch, polyvinyl alcohol, hydroxyethyl cellulose, methyl cellulose, gelatin, starch and cellulose-based cross-linked polymers, hydroxypropyl cellulose, hydroxypropyl methylcellulose, polyvinylpyrrolidone, crosslinked starch, microcrystalline cellulose, chitin, cellulose acetate, cellulose proprionate, cellulose acetate propionate, cellulose acetate butyrate, cellulose triacetate, aminoacryl-methacrylate copolymer (Eudragit® RS-PM, Rohm & Haas), pullulan, collagen, casein, agar, gum arabic, sodium carboxymethyl cellulose, carboxymethyl ethyl cellulose, swellable hydrophilic polymers, poly(hydroxyalkyl methacrylate) (m. wt. about 5 k-5,000 k), polyvinylpyrrolidone (m. wt. about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about.30 k-300 k), polysaccharides such as agar, acacia, karaya, tragacanth, algins and guar, polyacrylamides, Polyox® polyethylene oxides (m. wt. about 100 k-5,000 k), AquaKeep® acrylate polymers, diesters of polyglucan, crosslinked polyvinyl alcohol and poly N-vinyl-2-pyrrolidone, sodium starch glycollate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, poly(ethylene terphthalate), poly(vinyl isobutyl ether), polyurethane, polyethylene oxides (e.g. Polyox®, Union Carbide), methyl ethyl cellulose, ethylhydroxy ethylcellulose, cellulose acetate, ethylcellulose, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, ethyl acrylate-methyl methacrylate copolymer, sorbitan esters, polydimethyl siloxane, natural gums, lecithins, pectin, alginates, ammonia alginate, sodium, calcium, potassium alginates, propylene glycol alginate, agar, gums: arabic, karaya, locust bean, tragacanth, carrageens, guar, xanthan, scleroglucan and mixtures and blends thereof.

III. Nanoparticulate Clopidogrel and Aspirin Combination Compositions

The invention provides compositions comprising a clopidogrel and aspirin combination, or salts or derivatives thereof, and at least one surface stabilizer. The surface stabilizers can be adsorbed on, or associated with, the surface of the clopidogrel particles, aspirin particles, or a particle comprising clopidogrel and aspirin. Surface stabilizers especially useful herein preferably physically adhere on, or associate with, the surface of the active agent, but do not chemically react with the clopidogrel and aspirin particles or itself. Individually adsorbed molecules of the surface stabilizer are essentially free of intermolecular cross-linkages.

The present invention also includes compositions comprising a clopidogrel and aspirin combination, or salts or derivatives thereof, together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

A. Active Agent Particles

The compositions of the invention comprise nanoparticulate clopidogrel particles and aspirin, which can also be in a nanoparticulate size.

The clopidogrel particles can comprise clopidogrel or a salt or derivative thereof, such as clopidogrel bisulfate. The clopidogrel particles can be in a crystalline phase, semi-crystalline phase, amorphous phase, semi-amorphous phase, or a combination thereof.

The aspirin particles can comprise aspirin or a salt or derivative thereof. The aspirin particles can be in a crystalline phase, semi-crystalline phase, amorphous phase, semi-amorphous phase, or a combination thereof.

B. Surface Stabilizers

Combinations of more than one surface stabilizers can be used in the invention. For example, if aspirin is present in a nanoparticulate size, two different surface stabilizers can be used for the nanoparticulate clopidogrel and nanoparticulate aspirin. Alternatively, only one type of surface stabilizer may be used, even if both clopidogrel and aspirin are present in a nanoparticulate size. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, ionic, anionic, cationic, and zwitterionic surfactants or compounds.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₂₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quartemary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium- 18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

C. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

D. Nanoparticulate Clopidogrel and Aspirin Combination Particle Size

The compositions of the invention comprise nanoparticulate particles of clopidogrel, or a salt or derivative thereof, which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Optionally, the compositions of the invention comprise nanoparticulate particles of aspirin, or a salt or derivative thereof, which have an effective average particle size of less than about 2000 nm (i.e., 2 microns), less than about 1900 nm, less than about 1800 nm, less than about 1700 nm, less than about 1600 nm, less than about 1500 nm, less than about 1400 um, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the clopidogrel, or clopidogrel and aspirin combination with nanoparticulate aspirin, particles have a particle size of less than the effective average, by weight (or by other suitable measurement technique, such as by volume, number, etc.), i.e., less than about 2000 nm, 1900 nm, 1800 nm, etc., when measured by the above-noted techniques. In other embodiments of the invention, at least about 60%, at least about 70%, at least about 80%, at least about 90%, at least about 95% or at least about 99%, of the clopidogrel particles, aspirin particles, or a combination thereof, have a particle size of less than the effective average, i.e., less than about 2000 nm, 1900 nm, 1800 nm, 1700 nm, etc.

In the present invention, the value for D50 of a nanoparticulate clopidogrel composition, nanoparticulate aspirin composition, or a combination thereof is the particle size below which 50% of the clopidogrel particles and/or aspirin particles fall, by weight (or by other suitable measurement technique, such as by volume, number, etc.). Similarly, D90 is the particle size below which 90% of the clopidogrel particles and/or aspirin particles fall, by weight (or by other suitable measurement technique, such as by volume, number, etc.).

E. Concentration of Clopidogrel and Aspirin Combination and Surface Stabilizers

The relative amounts of clopidogrel and aspirin combination, or salts or derivatives thereof, and one or more surface stabilizers can vary widely. The optimal amount of the individual components can depend, for example, upon the particular clopidogrel and aspirin combination selected, the hydrophilic lipophilic balance (HLB), melting point, and the surface tension of water solutions of the stabilizer, etc.

In a first embodiment of the invention, the concentration of the clopidogrel and aspirin combination can vary from about 99.5% to about 0.001%, from about 95% to about 0.1%, or from about 90% to about 0.5%, by weight, based on the total combined dry weight of the clopidogrel and aspirin combination and at least one surface stabilizer, not including other excipients. The concentration of the at least one surface stabilizer can vary from about 0.5% to about 99.999%, from about 5.0% to about 99.9%, or from about 10% to about 99.5%, by weight, based on the total combined dry weight of the clopidogrel and aspirin combination and at least one surface stabilizer, not including other excipients.

In a second embodiment of the invention, the concentration of the clopidogrel can vary from about 99.5% to about 0.001%, from about 95% to about 0.1%, or from about 90% to about 0.5%, by weight, based on the dry weight of the clopidogrel and at least one surface stabilizer, not including other excipients. The concentration of the at least one surface stabilizer can vary from about 0.5% to about 99.999%, from about 5.0% to about 99.9%, or from about 10% to about 99.5%, by weight, based on the total dry weight of the clopidogrel and at least one surface stabilizer, not including other excipients.

In a third embodiment of the invention, the concentration of the aspirin can vary from about 99.5% to about 0.001%, from about 95% to about 0.1%, or from about 90% to about 0.5%, by weight, based on the dry weight of the aspirin and at least one surface stabilizer, not including other excipients. The concentration of the at least one surface stabilizer can vary from about 0.5% to about 99.999%, from about 5.0% to about 99.9%, or from about 10% to about 99.5%, by weight, based on the total dry weight of the aspirin and at least one surface stabilizer, not including other excipients.

F. Exemplary Nanoparticulate Clopidogrel Bisulfate and Aspirin Combination Tablet Formulations

Several exemplary clopidogrel bisulfate and aspirin combination tablet formulations are given below. These examples are not intended to limit the claims in any respect, but rather to provide exemplary tablet formulations of clopidogrel bisulfate and aspirin combination which can be utilized in the methods of the invention. Such exemplary tablets can also comprise a coating agent.

Exemplary Nanoparticulate
Clopidogrel Bisulfate and Aspirin Combination Tablet
Formulation #1

| Component | g/Kg |
|---|---|
| Clopidogrel Bisulfate and Aspirin | about 50 to about 500, each |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate
Clopidogrel Bisulfate and Aspirin Combination Tablet
Formulation #2

| Component | g/Kg |
|---|---|
| Clopidogrel Bisulfate and Aspirin | about 100 to about 300, each |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate
Clopidogrel Bisulfate and Aspirin Combination Tablet
Formulation #3

| Component | g/Kg |
|---|---|
| Clopidogrel Bisulfate and Aspirin | about 200 to about 225, each |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate
Clopidogrel Bisulfate and Aspirin Combination Tablet
Formulation #4

| Component | g/Kg |
|---|---|
| Clopidogrel Bisulfate and Aspirin | about 119 to about 224, each |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

IV. Methods of Making Nanoparticulate Clopidogrel and Aspirin Combination Compositions

The compositions comprising a nanoparticulate clopidogrel and aspirin combination, or salts or derivatives thereof, can be made using, for example, milling, homogenization, precipitation, freezing, or template emulsion techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate clopidogrel and aspirin combination compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

Aspirin can be reduced in size simultaneously with clopidogrel, or aspirin can be separately reduced in particle size (using the same or a different technique), and then the nanoparticulate aspirin composition can be combined with the nanoparticulate clopidogrel formulation to form a composition according to the invention. Alternatively, conventional microcrystalline aspirin can be added to nanoparticulate clopidogrel to form a composition according to the invention.

A. Milling to Obtain Nanoparticulate Clopidogrel and Aspirin Combination Dispersions

Milling a clopidogrel, and optionally aspirin, or salts or derivatives thereof, to obtain a nanoparticulate dispersion comprises dispersing the clopidogrel particles in a liquid dispersion medium in which the clopidogrel is poorly soluble, followed by applying mechanical means in the presence of grinding media to reduce the particle size of the clopidogrel to the desired effective average particle size. The dispersion medium can be, for example, water, safflower oil, ethanol, t-butanol, glycerin, polyethylene glycol (PEG), hexane, or glycol. A preferred dispersion medium is water.

The clopidogrel particles can be reduced in size in the presence of at least one surface stabilizer. Alternatively, clopidogrel particles can be contacted with one or more surface stabilizers after attrition. Other compounds, such as a diluent, can be added to the clopidogrel and aspirin combination/surface stabilizer composition during the size reduction process. Dispersions can be manufactured continuously or in a batch mode.

B. Precipitation to Obtain Nanoparticulate Clopidogrel and Aspirin Combination Compositions

Another method of forming the desired nanoparticulate clopidogrel, and optionally aspirin, or salts or derivatives thereof, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the clopidogrel and aspirin combination in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

C. Homogenization to Obtain Nanoparticulate Clopidogrel and Aspirin Combination Compositions

Exemplary homogenization methods of preparing nanoparticulate active agent compositions are described in U.S. Pat. No. 5,510,118, for “Process of Preparing Therapeutic Compositions Containing Nanoparticles.” Such a method comprises dispersing particles of a clopidogrel, and optionally aspirin, or salts or derivatives thereof, in a liquid dispersion medium, followed by subjecting the dispersion to homogenization to reduce the particle size of a clopidogrel to the desired effective average particle size. The clopidogrel particles can be reduced in size in the presence of at least one surface stabilizer. Alternatively, the clopidogrel particles can be contacted with one or more surface stabilizers either before or after attrition. Other compounds, such as a diluent, can be added to the clopidogrel/surface stabilizer composition either before, during, or after the size reduction process. Dispersions can be manufactured continuously or in a batch mode.

D. Cryogenic Methodologies to Obtain Nanoparticulate Clopidogrel and Aspirin Combination Compositions

Another method of forming the desired nanoparticulate clopidogrel, and optionally aspirin, or salts or derivatives thereof, composition is by spray freezing into liquid (SFL). This technology comprises an organic or organoaqueous solution of clopidogrel with stabilizers, which is injected into a cryogenic liquid, such as liquid nitrogen. The droplets of the clopidogrel and aspirin combination solution freeze at a rate sufficient to minimize crystallization and particle growth, thus formulating nanostructured clopidogrel particles. Depending on the choice of solvent system and processing conditions, the nanoparticulate clopidogrel particles can have varying particle morphology. In the isolation step, the nitrogen and solvent are removed under conditions that avoid agglomeration or ripening of the clopidogrel particles.

As a complementary technology to SFL, ultra rapid freezing (URF) may also be used to created equivalent nanostructured clopidogrel and aspirin combination particles with greatly enhanced surface area. URF comprises an organic or organoaqueous solution of clopidogrel with stabilizers onto a cryogenic substrate.

E. Emulsion Methodologies to Obtain Nanoparticulate Clopidogrel and Aspirin Combination Compositions

Another method of forming the desired nanoparticulate clopidogrel, and optionally aspirin, or salts or derivatives thereof, composition is by template emulsion. Template emulsion creates nanostructured clopidogrel particles with controlled particle size distribution and rapid dissolution performance. The method comprises an oil-in-water emulsion that is prepared, then swelled with a non-aqueous solution comprising the clopidogrel and stabilizers. The particle size distribution of the clopidogrel particles is a direct result of the size of the emulsion droplets prior to loading with the clopidogrel a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured clopidogrel particles are recovered. Various clopidogrel particles morphologies can be achieved by appropriate control of processing conditions.

IV. Controlled Release Nanoparticulate Clopidogrel and Aspirin Combination Formulations

Another aspect of the present invention comprises covering the nanoparticulate clopidogrel and aspirin combination particles described above in a polymeric coating or matrix. Since the solubility of clopidogrel and aspirin combination is pH-dependent, the dissolution rate and consequent bioavailability of the drug can change as it passes through different areas of the gastroenterologic system. Coating the particles for a sustained and/or controlled release results in an improved, consistent dissolution rate of the drug which will avoid the occurrence of localized high drug concentrations. One or both of the clopidogrel and aspirin may be coated.

Any coating material which modifies the release of the nanoparticulate clopidogrel and aspirin combination particles in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the Trade Mark Eudragite S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, Polyox® polyethylene oxides (m. wt. ,about.100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalateacetate trimaletate, hydroxy propyl methylcellulose phthalate, polyvinyl acetate phthalate, dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate and mixtures thereof.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of an active agent dispersed therein in vitro or in vivo. Modified release matrix materials suitable for the practice of the present invention include but are not limited to microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

V. Methods of Using the Nanoparticulate Clopidogrel and Aspirin Combination Compositions of the Invention

The invention provides a method of increasing bioavailability of a clopidogrel, or salts or derivatives thereof, in a subject. Such a method comprises orally administering to a subject an effective amount of a composition comprising a clopidogrel.

In one embodiment of the invention, the clopidogrel/aspirin composition, in accordance with standard pharmacokinetic practice, has a bioavailability that is about 50% greater, about 40% greater, about 30% greater, about 20% greater, or about 10% greater than a conventional dosage form.

The compositions of the invention are useful in the prevention and treatment of pathological states induced by platelet aggregation. Such pathological states include, but are not limited to, cardiovascular and cerebrovascular system diseases such as the thromboembolic disorders associated with atherosclerosis or with diabetes such as unstable angina, cerebral attack, restenosis following angioplasty, endarterectomy or fitting of metallic endovascular prostheses, with rethrombosis following thrombolysis, with infarction, with dementia of ischemic origin, with peripheral arterial diseases, with haemodialyses, with auricular fibrillations or during the use of vascular prostheses or aortocoronary bypasses or in relation to stable or unstable angor. Preferably, the compositions of the invention are useful in the prevention and treatment of cardiovascular disease.

The clopidogrel and aspirin combination, or salts or derivatives thereof, compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate clopidogrel and aspirin combination, or salts or derivatives thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato.or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a clopidogrel and aspirin combination, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

“Therapeutically effective amount” as used herein with respect to a clopidogrel and aspirin combination, dosage shall mean that dosage that provides the specific pharmacological response for which a clopidogrel and aspirin combination is administered in a significant number of subjects in need of such treatment. It is emphasized that therapeutically effective amount, administered to a particular subject in a particular instance will not always be effective in treating the diseases described herein, even though such dosage is deemed a ‘therapeutically effective amount’ by those skilled in the art. It is to be further understood that clopidogrel and aspirin combination dosages are, in particular instances, measured as oral dosages, or with reference to drug levels as measured in blood.

One of ordinary skill will appreciate that effective amounts of a clopidogrel and aspirin combination can be determined empirically and can be employed in pure form or, where such forms exist, in pharmaceutically acceptable salt, ester, or prodrug form. Actual dosage levels of a clopidogrel and aspirin combination in the nanoparticulate compositions of the invention may be varied to obtain an amount of a clopidogrel and aspirin combination that is effective to obtain a desired therapeutic response for a particular composition and method of administration. The selected dosage level therefore depends upon the desired therapeutic effect, the route of administration, the potency of the administered clopidogrel and aspirin combination, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following example is for illustrative purposes only, and should not be interpreted as restricting the spirit and scope of the invention, as defined by the scope of the claims that follow. All references cited herein, including U.S. patents, are specifically incorporated by reference.

### EXAMPLE 1

The purpose of this example was to describe how a nanoparticulate clopidogrel/aspirin composition could be prepared.

An aqueous dispersion of clopidogrel bisulfate can be combined with one or more surface stabilizers, followed by milling in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The composition can be milled for a suitable period of time, such as about 60 min. at a speed of 2500.

The milled composition can be harvested and analyzed via microscopy. Microscopy can be done, for example, using a Lecia DM5000B microscope and Lecia CTR 5000 light source (Laboratory Instruments and Supplies Ltd., Ashbourne Co., Meath, Ireland). Microscopy can show the presence of discrete clopidogrel nanoparticles.

The particle size of the milled clopidogrel particles can also be measured, in Milli Q Water, using a Horiba LA-910 Particle Sizer (Particular Sciences, Hatton Derbyshire, England). A composition having a D50 particle size of less than 2000 nm meets the criteria of the present invention.

Particle size can be measured initially and after 60 seconds of sonication. Particle sizes that vary significantly following sonication are undesirable, as it is indicative of the presence of clopidogrel aggregates. Such aggregates result in compositions having highly variable particle sizes. Such highly variable particle sizes can result in variable absorption between dosages of a drug, and therefore are undesirable.

The resultant nanoparticulate clopidogrel composition can be combined with conventional, microcrystalline aspirin, or nanoparticulate aspirin.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate clopidogrel and aspirin composition comprising:
(a) particles of a clopidogrel, or a salt or derivative thereof, having an effective average particle size of less than about 2000 nm;
(b) particles of aspirin, or a salt or derivative thereof; and
(c) at least one surface stabilizer.

2. The composition of claim 1, wherein the nanoparticulate clopidogrel is clopidogrel bisulfate.

3. The composition of claim 1, wherein the clopidogrel particles, aspirin particles, or a combination thereof are selected from the group consisting of a crystalline phase, an amorphous phase, a semi-crystalline phase, a semi amorphous phase, and mixtures thereof.

4. The composition of claim 1, wherein the aspirin particles have an effective average particle size of less than about 2000 nm.

5. The composition of claim 1, wherein the effective average particle size of the clopidogrel particles, aspirin particles, or both the clopidogrel and aspirin particles, are selected from the group consisting of less than about 1900 mn, less than about 1800 nm, less than about 1700 nm, less than about 1600 nm, less than about 1500 nm, less than about 1400 mn, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

6. The composition of claim 1, wherein the clopidogrel particles have improved bioavailability as compared to conventional clopidogrel tablets.

7. The composition of claim 1, wherein the composition is formulated:
(a) for administration selected from the group consisting of oral, pulmonary, rectal, colonic, parenteral, intracisternal, intravaginal, intraperitoneal, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, and mixed immediate release and controlled release formulations;
(d) any combination of (a), (b), and (c).

8. The composition of claim 1, wherein the composition further comprises one or more pharmaceutically acceptable excipients, carriers, or a combination thereof.

9. The composition of claim 1, wherein:
(a) clopidogrel, aspirin, or a combination thereof is present in an amount consisting of from about 99.5% to about 0.001%, from about 95% to about 0. 1%, and from about 90% to about 0.5%, by weight, based on the total combined dry weight of clopidogrel, aspirin, or a combination thereof, respectively, and at least one surface stabilizer, not including other excipients;
(b) at least one surface stabilizer is present in an amount of from about 0.5% to about 99.999% by weight, from about 5.0% to about 99.9% by weight, and from about 10% to about 99.5% by weight, based on the total combined dry weight of clopidogrel, aspirin, or a combination thereof, and at least one surface stabilizer, not including other excipients; or
(c) a combination thereof.

10. The composition of claim 1, wherein the surface stabilizer is selected from the group consisting of a nonionic surface stabilizer, an anionic surface stabilizer, a cationic surface stabilizer, a zwitterionic surface stabilizer, and an ionic surface stabilizer.

11. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

12. The composition of claim 1, additionally comprising one or more active agents useful for the prevention and treatment of a pathology induced by platelet aggregation.

13. The composition of claim 12, wherein the pathology is cardiovascular disease.

14. The composition of claim 12, wherein the one or more active agents is selected from the group consisting of calcium-entry blocking agents, antianginal agents, cardiac glycosides, vasodilators, antihypertensive agents, blood lipid-lowering agents, antidysrhythmic agents, and antithrombotic agents.

15. The composition of claim 1, wherein the composition does not produce significantly different absorption levels when administered under fed as compared to fasting conditions.

16. The composition of claim 1, wherein administration of the composition to a subject in a fasted state is bioequivalent to administration of the composition to a subject in a fed state.

17. The composition of claim 1 wherein the composition has:
(a) a Cₘₐₓ for clopidogrel, or a salt or derivative thereof, when assayed in the plasma of a mammalian subject following administration that is greater than the Cₘₐₓ for a non-nanoparticulate formulation of the same clopidogrel, or a salt or derivative thereof, administered at the same dosage;
(b) an AUC for clopidogrel, or a salt or derivative thereof, when assayed in the plasma of a mammalian subject following administration that is greater than the AUC for a non-nanoparticulate formulation of the same clopidogrel, or a salt or derivative thereof, administered at the same dosage;
(c) a Tₘₐₓ for clopidogrel, or a salt or derivative thereof, when assayed in the plasma of a mammalian subject following administration that is less than the Tₘₐₓ for a non-nanoparticulate formulation of the same clopidogrel, or a salt or derivative thereof, administered at the same dosage; or
(d) any combination of (a), (b), and (c).

18. A controlled release pharmaceutical composition comprising the clopidogrel and aspirin combination composition of claim 1, wherein the clopidogrel particles, aspirin particles, or a combination thereof are covered with one or more layers of a polymeric coating.

19. A controlled release pharmaceutical composition comprising the clopidogrel and aspirin combination composition of claim 1, wherein the particles are incorporated in a polymeric matrix.

20. The composition of claim 1, further comprising an enteric coating encasing the clopidogrel particles, aspirin particles, or a combination thereof.

21. A method of preparing a nanoparticulate clopidogrel and aspirin combination, comprising:
(a) contacting particles of clopidogrel, or a salt or derivative thereof, with at least one surface stabilizer for a time and under conditions sufficient to provide a nanoparticulate clopidogrel composition having an effective average particle size of less than about 2000 nm, and;
(b) combining the resultant nanoparticulate clopidogrel with aspirin, or a salt or derivative thereof.

22. A method for reducing irritancy of the stomach and/or esophagus, minimizing solubilization and reducing of precipitation of clopidogrel, with the administration of an oral clopidogrel and aspirin combination, comprising the administration of the composition of claim 1.

23. A stable nanoparticulate clopidogrel composition comprising:
(a) particles of a clopidogrel, or a salt or derivative thereof, having an effective average particle size of less than about 2000 nm;
(b) at least one surface stabilizer; and
(c) an enteric coating encasing the clopidogrel particles.

24. A method for reducing irritancy of the stomach and/or esophagus, minimizing solubilization and reducing of precipitation of clopidogrel, with the administration of an oral clopidogrel, comprising the administration of the composition of claim 23.

25. A composition comprising:
(a) a clopidogrel, or a salt or derivative thereof; and
(b) an enteric coating encasing the clopidogrel for inhibiting release of the clopidogrel to the stomach.

26. The composition of claim 25, wherein the amount of clopidogrel released into the stomach of a subject, relative to the total dose administered to the subject, is selected from the group consisting of no more than about 0.05%, no more than about 0.5%, no more than about 1%, no more than about 5% and no more than about 10%.

27. The composition of claim 25, wherein the amount of clopidogrel released in the intestine of a subject, relative to the total dose administered to the subject, is selected from the group consisting of at least about 90%, at least about 95%, at least about 97% and at least about 100%.

28. A method for reducing irritancy of the stomach and/or esophagus, minimizing solubilization and reducing of precipitation of clopidogrel, with the administration of an oral clopidogrel, comprising the administration of the composition of claim 25.
